(19) United States
(12) Patent Application Publication
(10) Pub. No.: US 2023/0087973 A1
(43) Pub. Date: Mar. 23, 2023

(54) VENTILATION ADJUSTMENT METHOD AND HIGH-FREQUENCY VENTILATION SYSTEM

(71) Applicant: SHENZHEN MINDRAY BIO-MEDICAL ELECTRONICS CO., LTD., Shenzhen (CN)

(72) Inventors: Leping WU, Shenzhen (CN); Xiaoyong ZHOU, Shenzhen (CN); Kun CAI, Shenzhen (CN); Yang XIAO, Shenzhen (CN)

(73) Assignee: SHENZHEN MINDRAY BIO-MEDICAL ELECTRONICS CO., LTD., Shenzhen (CN)

(21) Appl. No.: 17/991,804

(22) Filed: Nov. 21, 2022

Related U.S. Application Data
(63) Continuation-in-part of application No. PCT/CN2020/091226, filed on May 20, 2020.

(51) Int. Cl. A61M 16/00

(52) U.S. Cl. CPC A61M 16/0096; A61M 16/024; A61M 2016/1025

(57) ABSTRACT

A ventilation adjustment method and a high-frequency ventilation system, which ensure stable and accurate oxygen concentration control within an oxygen concentration setting range, are disclosed. The ventilation adjustment method includes: determining a first gas flow rate control value and a second gas flow rate control value according to a target output flow rate and an oxygen concentration setting value; determining whether the first gas flow rate control value falls into a first dead zone range and whether the second gas flow rate control value falls into a second dead zone range; if the first gas flow rate control value falls into the first dead zone range, maintaining a first gas flow rate controller turned on in an expiratory phase; and if the second gas flow rate control value falls into the second dead zone range, maintaining a second gas flow rate controller turned on in the expiratory phase.

### CROSS-REFERENCE TO RELATED APPLICATIONS

This application is a bypass continuation-in-part application of Patent Cooperation Treaty Application No. PCT/CN2020/091226, filed May 20, 2020, the content of which is incorporated herein by reference in its entirety.

### TECHNICAL FIELD

The disclosure relates to the technical field of respiratory assistance, and more particularly to a ventilation adjustment method and a high-frequency ventilation system.

### BACKGROUND

As an important respiratory support technology, mechanical ventilation has been widely used in clinical treatment. Mechanical ventilation can be divided into conventional mechanical ventilation (CMV) and high-frequency ventilation (HFV) according to the ventilation frequency. At present, the clinical ventilation treatment is still dominated by conventional mechanical ventilation, which plays an important role in correcting severe hypoxemia, hypercapnia, relieving high-frequency ventilation system fatigue, etc. In recent years, with the update and improvement of high-frequency ventilation treatment technology, high-frequency ventilation has become an important supplement to conventional mechanical ventilation, and high-frequency ventilation also plays an increasingly important role.

High-frequency ventilation system can be divided into two types according to the implementation principle, one is diaphragm or piston type, and the other is valve-controlled type. Both implementation methods generate high-frequency pressure oscillation. However, a control pressure of the valve-controlled high-frequency ventilation system can be selected to different ranges according to a measurement range of a proportional valve, so it has stronger oscillation ability and can be used in a wider range.

Valve-controlled high-frequency ventilation needs to generate a pulsed gas flow by quickly opening and closing of the valve to achieve the desired high-frequency oscillating pressure. In addition, an oxygen concentration of the high-frequency ventilation system is also controlled by a flow rate of the proportional valve.

However, due to the viscosity of the proportional valve, there is a dead zone at a small flow rate. The common ventilation frequency of high-frequency ventilation is as high as 300-1200 times/min. During the pressure oscillation process, the flow rate of the proportional valve should be controlled quickly. If the conventional control method is used during the high-frequency ventilation process, the proportional valve cannot continuously provide a stable flow rate when the flow rate is close to the dead zone. As a result, when the oxygen concentration is arranged below 40% or above 80%, the oxygen concentration fluctuates.

### SUMMARY

Embodiments of this disclosure provide a ventilation adjustment method and a high-frequency ventilation system, which are configured to generate a stable small flow rate during a high-frequency vibration, so as to ensure stable and accurate oxygen concentration control within an oxygen concentration setting range.

According to a first aspect, an embodiment of this disclosure provides a ventilation adjustment method which is applied to a high-frequency ventilation system which includes a gas source interface, an inspiratory branch, a ventilation control device, and a high-frequency pressure reduction module; the inspiratory branch includes a first gas branch, a second gas branch, a mixing branch, a first gas flow rate controller which generates a high-frequency pulse flow rate and is arranged at the first gas branch, and a second gas flow rate controller which generates a high-frequency pulse flow rate and is arranged at the second gas branch; wherein the ventilation adjustment method includes:

determining a first gas flow rate control value and a second gas flow rate control value according to a target output flow rate and an oxygen concentration setting value;

determining whether the first gas flow rate control value falls into a first dead zone range and determining whether the second gas flow rate control value falls into a second dead zone range;

maintaining the first gas flow rate controller turn-on, if the first gas flow rate control value falls into the first dead zone range; and

maintaining the second gas flow rate controller turn-on, if the second gas flow rate control value falls into the second dead zone range;

wherein the first dead zone range corresponds to a dead zone range of the first gas flow rate controller, and the second dead zone range corresponds to a dead zone range of the second gas flow rate controller.

Optionally, the method further includes:

controlling the second gas flow rate controller to generate high-frequency oscillation, if the first gas flow rate control value falls into the first dead zone range and the second gas flow rate control value falls outside the second dead zone range;

controlling the first gas flow rate controller to generate high-frequency oscillation, if the first gas flow rate control value falls outside the first dead zone range and the second gas flow rate control value falls into the second dead zone range.

Optionally, the method further includes:

controlling the high-frequency pressure reduction module to generate high-frequency oscillation, if the first gas flow rate control value falls into the first dead zone range and the second gas flow rate control value falls into the second dead zone range.

Optionally, the high-frequency pressure reduction module includes a high-frequency valve and a turbine.

Optionally, controlling the high-frequency pressure reduction module to generate high-frequency oscillation, includes:

controlling the high-frequency valve and the turbine to generate high-frequency oscillation according to a preset high-frequency oscillation frequency.

Optionally, the method further includes:

controlling the first gas flow rate controller and the second gas flow rate controller to generate a high-frequency pulse flow rate, if the first gas flow rate control value falls outside the first dead zone range and the second gas flow rate control value falls outside the second dead zone range.

Optionally, the inspiratory branch is further provided with an oxygen concentration detector, which is configured to detect an oxygen concentration of an output gas of the inspiratory branch;

wherein the method further includes:

adjusting the first gas flow rate controller and the second gas flow rate controller according to the oxygen concentration of the output gas and a target oxygen concentration, if the oxygen concentration of the output gas which is detected by the oxygen concentration detector fails to reach the target oxygen concentration.

Optionally, adjusting the first gas flow rate controller and the second gas flow rate controller according to the oxygen concentration of the output gas and a target oxygen concentration, includes:

adjusting the first gas flow rate controller and the second gas flow rate controller according to the oxygen concentration of the output gas and the target oxygen concentration, based on a preset adjustment rule.

Optionally, the method further includes:

determining the first dead zone range according to a flow rate-electric current curve of the first gas flow rate controller; and

determining the second dead zone range according to a flow rate-electric current curve of the second gas flow rate controller.

Optionally, the method further includes:

obtaining the flow rate-electric current curve of the first gas flow rate controller and the flow rate-electric current curve of the second gas flow rate controller.

According to a second aspect, an embodiment of this disclosure provides a high-frequency ventilation system which includes a gas source interface, an inspiratory branch, a ventilation control device, and a high-frequency pressure reduction module; the inspiratory branch includes a first gas branch, a second gas branch, a mixing branch, a first gas flow rate controller which generates a high-frequency pulse flow rate and is arranged at the first gas branch, and a second gas flow rate controller which generates a high-frequency pulse flow rate and is arranged at the second gas branch; wherein the ventilation control device is configured to:

determine a first gas flow rate control value and a second gas flow rate control value according to a target output flow rate and an oxygen concentration setting value;

determine whether the first gas flow rate control value falls into a first dead zone range and deteimine whether the second gas flow rate control value falls into a second dead zone range;

maintain the first gas flow rate controller turn-on, if the first gas flow rate control value falls into the first dead zone range; and

maintain the second gas flow rate controller turn-on, if the second gas flow rate control value falls into the second dead zone range;

wherein the first dead zone range corresponds to a dead zone range of the first gas flow rate controller, and the second dead zone range corresponds to a dead zone range of the second gas flow rate controller.

Optionally, the ventilation control device is further configured to:

control the second gas flow rate controller to generate high-frequency oscillation, if the first gas flow rate control value falls into the first dead zone range and the second gas flow rate control value falls outside the second dead zone range;

control the first gas flow rate controller to generate high-frequency oscillation, if the first gas flow rate control value falls outside the first dead zone range and the second gas flow rate control value falls into the second dead zone range.

Optionally, the ventilation control device is further configured to:

control the high-frequency pressure reduction module to generate high-frequency oscillation, if the first gas flow rate control value falls into the first dead zone range and the second gas flow rate control value falls into the second dead zone range.

Optionally, the high-frequency pressure reduction module includes a high-frequency valve and a turbine.

Optionally, in order to control the high-frequency pressure reduction module to generate high-frequency oscillation, the ventilation control device is further configured to:

control the high-frequency valve and the turbine to generate the high-frequency oscillation according to a preset high-frequency oscillation frequency.

Optionally, the ventilation control device is further configured to:

control the first gas flow rate controller and the second gas flow rate controller to generate a high-frequency pulse flow rate, if the first gas flow rate control value falls outside the first dead zone range and the second gas flow rate control value falls outside the second dead zone range.

Optionally, the inspiratory branch is further provided with an oxygen concentration detector, which is configured to detect an oxygen concentration of an output gas of the inspiratory branch; wherein the ventilation control device is further configured to:

adjust the first gas flow rate controller and the second gas flow rate controller according to the oxygen concentration of the output gas and a target oxygen concentration, if the oxygen concentration of the output gas which is detected by the oxygen concentration detector fails to reach the target oxygen concentration.

Optionally, in order to adjust the first gas flow rate controller and the second gas flow rate controller according to the oxygen concentration of the output gas and a target oxygen concentration, wherein the ventilation control device is further configured to:

adjust the first gas flow rate controller and the second gas flow rate controller according to the oxygen concentration of the output gas and the target oxygen concentration, based on a preset adjustment rule.

Optionally, the ventilation control device is further configured to:

determine the first dead zone range according to a flow rate-electric current curve of the first gas flow rate controller; and

determine the second dead zone range according to a flow rate-electric current curve of the second gas flow rate controller.

Optionally, the ventilation control device is further configured to:

obtain the flow rate-electric current curve of the first gas flow rate controller and the flow rate-electric current curve of the second gas flow rate controller.

According to a third aspect, an embodiment of this disclosure provides a computer-readable storage medium in which instructions are stored, wherein when running the computer-readable storage medium on a computer, the computer executes the ventilation adjustment method provided in the first aspect.

To sum up, it can be seen that in the embodiment provided by this disclosure, the first gas flow rate control value and the second gas flow rate control value are determined according to the target output flow rate and oxygen concentration setting;

whether the first gas flow rate control value falls into the first dead zone range and whether the second gas flow rate control value falls into the second dead zone range are determined respectively; if the first gas flow rate control value falls into the first dead zone, the first gas flow rate controller maintains turn-on in the expiratory phase, while if the second gas flow rate control value falls into the second dead zone, the second gas flow rate controller maintains turn-on in the expiratory phase, so that when the flow rate of the proportional valve is within its corresponding dead zone, the flow rate of the proportional valve can be adjusted to produce a stable small flow rate in the high-frequency oscillation process, which ensures stable and accurate oxygen concentration control within the oxygen concentration setting range.

### BRIEF DESCRIPTION OF THE DRAWINGS

FIG. 1 is a structural diagram of a high-frequency ventilation system provided by an embodiment of this disclosure.

FIG. 2 is another structural diagram of a high-frequency ventilation system provided by an embodiment of this disclosure.

FIG. 3 is a control effect diagram when employing a conventional oxygen mixing control algorithm provided by an embodiment of this disclosure.

FIG. 4 is a control effect diagram when employing a ventilation adjustment method provided by an embodiment of this disclosure.

FIG. 5 is a diagram showing a flow rate control for dead zone and non-dead zone of a proportional valve provided by an embodiment of this disclosure.

FIG. 6 is a flow diagram of a ventilation adjustment method provided by an embodiment of this disclosure.

FIG. 7 is a virtual structure diagram of a high-frequency ventilation system provided by an embodiment of this disclosure.

### DETAILED DESCRIPTIONS

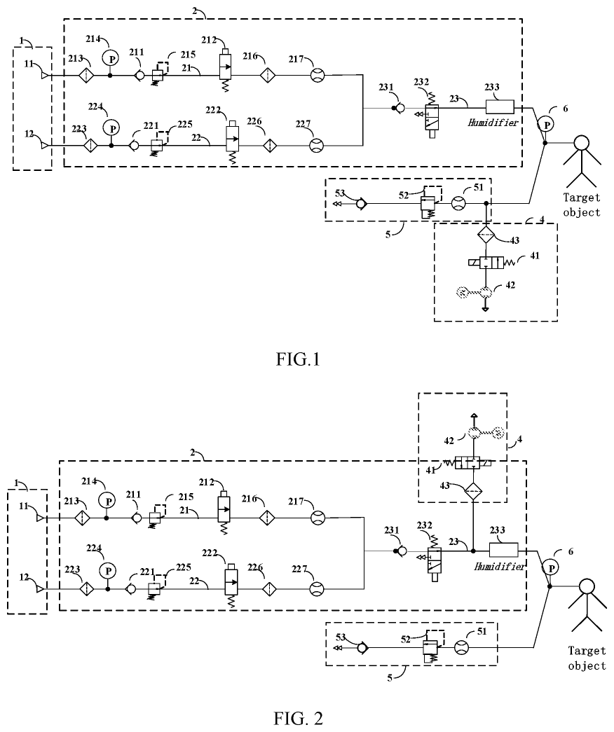

The terms “first”, “second”, “third”, “fourth” (if any presents), etc., in the specification, claims and attached drawings of this disclosure, are operable to distinguish different objects, rather than to describe a specific order. It should be understood that the objects described by these terms can be interchanged when appropriate, so that the embodiments described herein can be implemented in an order other than what is illustrated or described herein. In addition, the teams “include” and “have” and any variations thereof are intended to contain non-exclusive inclusions. For example, a process, method, system, product or device that includes a series of steps or units, is not necessarily limited to those steps or units that are clearly listed, but can include other steps or units that are not clearly listed or are inherent to these processes, methods, products or device. FIG. 1 is a structural diagram of a high-frequency ventilation system provided by an embodiment of this disclosure. FIG. 2 is another structural diagram of a high-frequency ventilation system provided by an embodiment of this disclosure. As shown in FIG. 1 and FIG. 2, the high-frequency ventilation system mainly includes a gas source interface 1, an inspiratory branch 2, a ventilation control device 3 (not shown) and a high-frequency pressure reduction module 4.

The inspiratory branch 2 is respectively connected with the gas source interface 1 and a patient pipeline which is connected with a respiratory system of a user.

The ventilation control device 3 is connected with the inspiratory branch 2 and the high-frequency pressure reduction module 4. During the inspiratory phase, the ventilation control device 3 controls a gas in the inspiratory branch 2 to generate high-frequency oscillation, and outputs the generated high-frequency oscillation gas through the inspiratory branch 2 and the patient pipeline. During the expiratory phase, the high-frequency pressure reduction module 4 actively extracts a gas exhaled by the user through the patient pipeline.

It should be noted that in an embodiment of this disclosure, the inspiratory branch 2 of the high-frequency ventilation system is configured to provide a gas transmission path in the inspiratory phase.

It should be noted that in the embodiment of this disclosure, as shown in FIG. 1 and FIG. 2, the high-frequency pressure reduction module 4 can include a high-frequency valve 41 and a turbine 42 to generate high-frequency oscillation for the exhaled gas of the patient. The specific high-frequency valve 41 can be any one of a proportional solenoid valve, a block valve and a servo valve, which is not limited herein. The high-frequency pressure reduction module 4 can also generate high-frequency oscillation through the first gas flow rate controller 212 and the second gas flow rate controller 222 in the inspiratory phase, which can be specifically adjusted according to the actual situation. In the expiratory phase, the ventilation control device 3 controls the high-frequency pressure reduction module 4 to actively extract the exhaled gas of the user according to the preset high-frequency oscillation frequency, so as to realize active exhalation.

It should be noted that in the embodiment of this disclosure, the medical staff can determine the preset high-frequency oscillation frequency according to the actual ventilation demand of the user, and the specific preset high-frequency oscillation frequency can be 3-50 Hz. Of course, it can also be set according to the actual situation of the user, without specific limitation.

In addition, it can be understood that the pipeline which is connected to the target object can also be provided with a third pressure sensor 6, which is connected with an output end of the inspiratory branch 2 and an input end of the expiratory branch 5.

It can be understood that in an embodiment of this disclosure, the high-frequency ventilation system further includes an expiratory branch 5, which is configured to provide an expiratory path in the expiratory phase.

It should be noted that in an embodiment of this disclosure, the high-frequency pressure reduction module 4 includes a high-frequency valve 41 and/or an electric gas extraction device 42, wherein, the high-frequency valve 41 can be any one of a proportional solenoid valve, a block valve and a servo valve, the electric gas extraction device 42 can be a turbine and other devices. During the expiratory phase, the ventilation control device 3 can control the turbine to rotate based on a preset high-frequency oscillation frequency. By controlling a rotation rate of the turbine, a negative force can be controlled to generate active exhalation. The turbine actively extracts the exhaled gas of the user to generate active exhalation. The specific high-frequency valve 41 and electric gas extraction device 42 can be selected according to the actual situation, and the embodiments of this disclosure are not limited this.

In an embodiment of this disclosure, as shown in FIG. 1, the high-frequency pressure reduction module 4 can be arranged at the expiratory branch 5 of the high-frequency ventilation system. As shown in FIG. 2, the high-frequency pressure reduction module 4 can also be arranged at the inspiratory branch 2 of the high-frequency ventilation system. In addition, as shown in FIGS. 1 and 2, the high-frequency pressure reduction module 4 includes not only the high-frequency valve 41 and the electric gas extraction device 42, but also an expiratory filter 43. In the expiratory phase, the ventilation control device 3 can control the high-frequency valve 41 to open, and control a rotation rate of the electric gas extraction device 42, such as the turbine, by adjusting an electric current or voltage, so as to extract the exhaled gas of the user through the expiratory filter 43. In addition, in the expiratory phase, the ventilation control device 3 can also control an expiratory valve of the expiratory branch 5 to open and exhaust gas simultaneously, thereby assisting the electric gas extraction device 42 to exhaust gas together. It should be noted that in the embodiments of this disclosure, according to different actual situations, just the high-frequency valve 41 can be opened to implement active exhalation, or just the electric gas extraction device 42 can be configured to implement active exhalation, or both of the high-frequency valve 41 and the electric gas extraction device 42 can be configured to implement active exhalation at the same time, and the embodiments of this disclosure are not limited for these. In order to facilitate understanding, the turbine is taken as an example of the electric gas extraction device 42 for explanation.

It should be noted that in the embodiment of this disclosure, as shown in FIGS. 1 and 2, the expiratory branch 5 may include an expiratory flow sensor 51, an expiratory valve 52 and an expiratory check valve 53. Wherein, the expiratory flow sensor 51 is connected with the patient pipeline to monitor a flow amount and tidal volume of the exhaled gas of the user. The expiratory valve 52 is connected with the expiratory flow sensor 51, which is configured to control an expiratory end pressure of the exhaled gas of the user and prevent collapse of the pulmonary alveoli after the user exhales. The expiratory check valve 53 is connected with the expiratory valve 52 to prevent gas from entering the expiratory branch.

It should be noted that in the embodiment of this disclosure, in the expiratory phase, when a normal frequency ventilation is adopted, the ventilation control device 3 controls the high-frequency valve 41 to close, and the exhaled gas of the user passes through the expiratory flow sensor 51 of the expiratory branch 5 and is discharged through the expiratory valve 52.

It can be understood that in an embodiment of this disclosure, the actual state of the user may not need to realize active exhalation in the expiratory phase. Therefore, the ventilation control device 3 can also control the active exhalation device 5 to close in the expiratory phase, so that the gas exhaled by the user from the patient pipeline can be discharged through the expiratory branch 5.

In an embodiment of this disclosure, as shown in FIGS. 1 and 2, the gas source interface 1 includes a first gas source interface 11 and a second gas source interface 12, the inspiratory branch 2 includes a first gas branch 21, a second gas branch 22 and a mixing branch 23.

A gas outlet end of the first gas branch 21 and a gas outlet end of the second gas branch 22 are respectively connected with a gas inlet end of the mixing branch 23.

A gas outlet end of the mixing branch 23 is connected with the patient pipeline.

The gas inlet end of the first gas branch 21 is connected with the first gas source interface 11.

The gas inlet end of the second gas branch 22 is connected with the second gas source interface 12.

It should be noted that, in an embodiment of this disclosure, as shown in FIGS. 1 and 2, the first gas source interface 11 is connected with an oxygen source, and the second gas source interface 12 is connected with an air source. Of course, the first gas source interface 11 can also be connected the air source, and the second gas source interface 12 can also be connected with the oxygen source, which is not limited in the embodiments of this disclosure.

Specifically, in an embodiment of this disclosure, as shown in FIGS. 1 and 2, the first gas branch 21 includes a first inspiratory check valve 211 and a first gas flow rate controller 212 which are connected in sequence, the second gas branch 22 includes a second inspiratory check valve 221 and a second gas flow rate controller 222 which are connected in sequence, and the mixing branch 23 includes a third inspiratory check valve 231 which is connected in sequence with the first gas branch 21 and the second gas branch 22.

The first inspiratory check valve 211 is connected with the first gas source interface 11, and the second inspiratory check valve 221 is connected with the second gas source interface 12.

The first gas flow rate controller 212 and the second gas flow rate controller 222 are respectively connected with the ventilation control device 3.

It should be noted that in the embodiment of this disclosure, as shown in FIGS. 1 and 2, the first gas branch 21 can include not only the first inspiratory check valve 211 and the first gas flow rate controller 212, but also a first filter 213, a first pressure sensor 214, a first pressure regulation valve 215, a second filter 216 and a first flow sensor 217. In addition, the second gas branch can include not only the second inspiratory check valve 221 and the second gas flow rate controller 222, but also a third filter 223, a second pressure sensor 224, a second pressure regulation valve 225, a fourth filter 226 and a second flow sensor 227.

Specifically, in an embodiment of this disclosure, as shown in FIGS. 1 and 2, in the first gas branch 21, the first filter 213 is connected with the first gas source interface 11 to prevent impurities from flowing into the downstream of the gas path and protect the downstream devices. The first pressure sensor 214 is connected with the first filter 213 and is configured to monitor a pressure of oxygen which is inputted through the first gas source interface 11, so as to send out an alarm when the pressure exceeds a maximum threshold value or is lower than a minimum threshold value. The first inspiratory check valve 211 is connected with the first pressure sensor 214 to prevent air from entering the branch. In additional, when just the second gas branch 22 is opened, reverse leakage of air entering the second gas branch 22 can be avoided. The first pressure regulation valve 215 is connected with the first inspiratory check valve 211, which can stabilize an input pressure of the gas source and ensure an accurate control of a downstream flow volume and pressure. The first gas flow rate controller 212 is connected with the first pressure regulation valve 215 for regulating and controlling a flow volume of the oxygen. The second filter 216 is connected with the first gas flow rate controller 212 and the first flow sensor 217. The second filter is configured to further purify the inputted oxygen, protect the downstream first flow sensor 217 for accurate measurement of the flow volume of the oxygen, and also to play a role in stabilizing the flow rate.

Specifically, in an embodiment of this disclosure, as shown in FIGS. 1 and 2, in the second gas branch 22, the third filter 223 is connected with the second gas source interface 12 to prevent impurities from flowing into the downstream of the gas path and protect the downstream devices. The second pressure sensor 224 is connected with the third filter 223 to monitor a pressure of an air which is inputted through the second gas source interface 12, so as to send out an alarm when the pressure exceeds a maximum threshold or is lower than a minimum threshold. The second inspiratory check valve 221 is connected with the second pressure sensor 224 to prevent oxygen from entering the branch. In additional, when just the first gas branch 21 is opened, reverse leakage of oxygen entering the first gas branch 21 can be avoided. The second pressure regulation valve 225 is connected with the second inspiratory check valve 221, which can stabilize the input pressure of the air source and ensure the accurate control of the downstream flow volume and pressure. The second gas flow rate controller 222 is connected with the second pressure regulation valve 225 to regulate and control a flow volume of the air. The fourth filter 226 is connected with the second gas flow rate controller 222 and the second flow sensor 227. The fourth filter 226 is configured to further purify the inputted air, protect the downstream second flow sensor 227 for accurate measurement of the flow volume of the oxygen, and also to play a role in stabilizing the flow rate.

It can be understood that in the embodiment of this disclosure, the first gas flow rate controller 212 and the second gas flow rate controller 222 respectively control the flow volume of oxygen and air, so that when oxygen and air are mixed in the mixing branch 23 to obtain mixed gas, the oxygen concentration in the mixed gas can be controlled to satisfy the ventilation needs of different users.

It should be noted that in an embodiment of this disclosure, the mixing branch 23 shown in FIGS. 1 and 2 not only includes the third inspiratory check valve 231, but further includes a safety valve 232 and a humidifier 233.

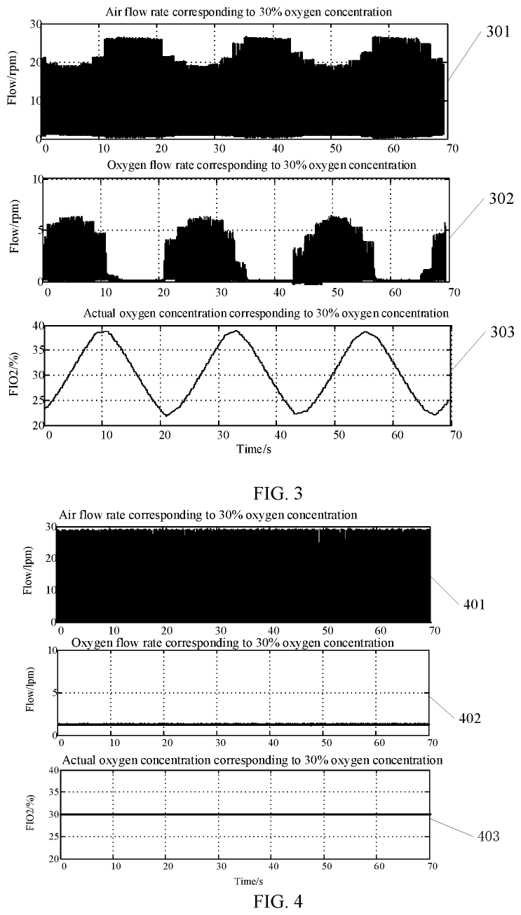

Please refer to FIG. 3, which is a control effect diagram when employing a conventional oxygen mixing control algorithm provided by an embodiment of this disclosure. When the target oxygen concentration is set as 30% during the high-frequency ventilation, the flow rate control cannot be stabilized due to the dead zone characteristics of the air proportional valve, resulting in the fluctuation of the actual oxygen concentration at the patient end by about 13%, which exceeds the accuracy requirement of the oxygen concentration required by the clinical requirements (the commonly used clinical accuracy range is 3%). As shown in FIG. 3, 301 represents the air flow rate diagram corresponding to 30% oxygen concentration, 302 represents the oxygen flow rate diagram corresponding to 30% oxygen concentration, and 303 represents the fluctuation diagram of actual oxygen concentration corresponding to 30% oxygen concentration, wherein no matter the air flow rate corresponding to 30% oxygen concentration, the oxygen flow rate corresponding to 30% oxygen concentration, or the actual oxygen concentration corresponding to 30% oxygen concentration, they all have fluctuations in varying degrees.

Please refer to FIG. 4, which is a control effect diagram when employing a ventilation adjustment method provided by an embodiment of this disclosure. For the small flow rate oxygen mixing fluctuation caused by the dead zone characteristics of the proportional valve in the high-frequency ventilation mode, the ventilation adjustment method in this disclosure keeps the proportional valve open when a proportional valve is within its corresponding dead zone, which ensures a stable small flow rate in the high-frequency oscillation process, ensures stable and accurate oxygen concentration control within the range of 21%-40% and 80%-100% oxygen concentration settings, and ensures that the oxygen concentration control accuracy is within 3%. Refer to FIG. 4, wherein 401 represents the air flow rate diagram corresponding to 30% oxygen concentration, 402 represents the oxygen flow rate corresponding to 30% oxygen concentration, and 403 represents the fluctuation diagram of actual oxygen concentration corresponding to 30% oxygen concentration.

Referring FIG. 4, no matter the air flow rate corresponding to 30% oxygen concentration, the oxygen flow rate corresponding to 30% oxygen concentration, or the actual oxygen concentration corresponding to 30% oxygen concentration, all of which have relatively stable oxygen flow rate, air flow rate and actual oxygen concentration when employing the ventilation adjustment method provided by an embodiment of this disclosure.

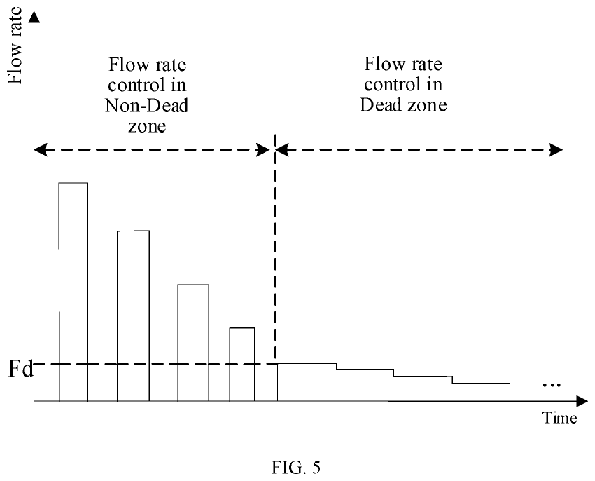

As shown in FIG. 5, when the oxygen concentration is set around 21% and 100%, the air proportional valve (the second gas flow rate controller in FIG. 1 and FIG. 2) or the oxygen proportional valve (the first gas flow rate controller in FIG. 1 and FIG. 2) cannot give a stable small target flow rate, and the phenomenon of periodic sudden opening and closing occurs, while the unstable control of the flow rate eventually causes the oxygen concentration to fluctuate. In fact, the periodic opening and closing of the proportional valve is caused by the dead zone of the proportional valve. Within the dead zone, the response of the proportional valve is nonlinear, and there is a fluctuation phenomenon. During the high-frequency ventilation, when the oxygen concentration is set around 21% or 100%, there must be a proportional valve to give a smaller control flow rate. During the feedback adjustment according to the oxygen concentration for the small flow rate, the nonlinear effect of the valve dead zone causes the proportional valve to close suddenly when the smaller flow rate is adjusted to near or within the dead zone. Theoretically, if the proportional valve is suddenly closed, it will gradually open again under the feedback adjustment of oxygen concentration. Due to the influence of the dead zone, the opening flow rate of the proportional valve is discontinuous, and the proportional valve suddenly opens when there is a flow rate. It just is the sudden opening and closing of the proportional valve during the control of small flow rate that causes the fluctuation of oxygen concentration. In fact, viscosity and dead zone are inherent characteristics of proportional valve, which cannot be eliminated fundamentally in control, and can only be compensated to reduce the influence of dead zone on control of proportional valve. However, for high-frequency ventilation, the control frequency is as high as 300-1200 times/min, and it is difficult to compensate the dead zone of the proportional valve in a short control period, which leads to more obvious dead zone characteristics of the valve.

In view of this, this disclosure provides a ventilation adjustment method. During the high-frequency ventilation, when the air proportional valve or oxygen proportional valve is adjusted within the dead zone in the inspiratory phase, the electric current control flow rate is maintained without closing the valve in the expiratory phase, so as to avoid the influence of the dead zone of the proportional valve on the control of small flow rate, and to ensure a relatively stable oxygen flow rate, air flow rate and relatively stable oxygen concentration.

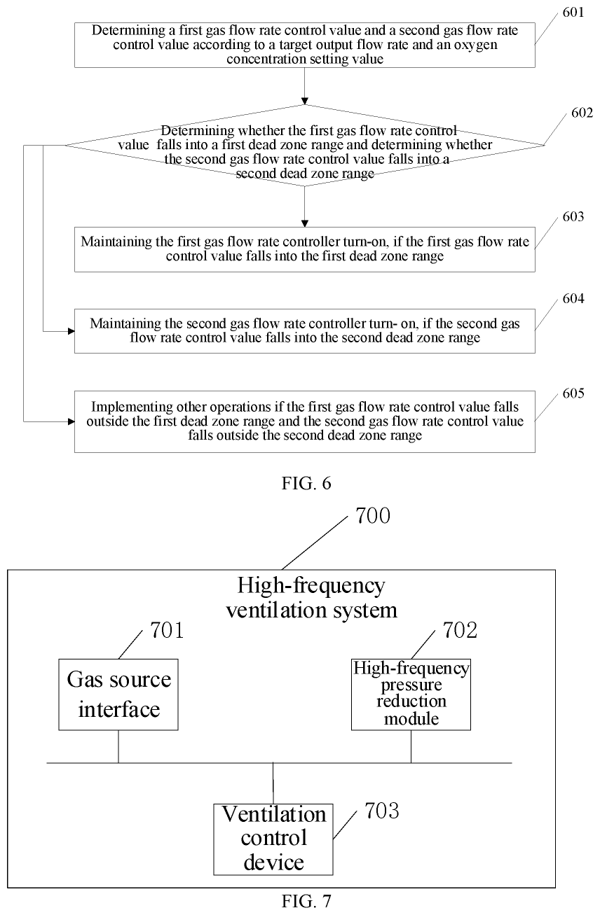

The ventilation adjustment method provided by an embodiment of this disclosure is described below with reference to FIG. 6.

Please refer to FIG. 6, which is a flow diagram of a ventilation adjustment method provided by an embodiment of this disclosure. The ventilation adjustment method is applied to a high-frequency ventilation system. The high-frequency ventilation system includes a gas source interface, an inspiratory branch, a ventilation control device and a high-frequency pressure reduction module. The inspiratory branch includes a first gas branch, a second gas branch, a mixing branch, a first gas flow rate controller which generates a high-frequency pulse flow rate and is arranged at the first gas branch, and a second gas flow rate controller which generates a high-frequency pulse flow rate and is arranged at the second gas branch; wherein the ventilation adjustment method includes following steps.

In step 601, a first gas flow rate control value and a second gas flow rate control value are determined according to a target output flow rate and an oxygen concentration setting value.

In this embodiment, the ventilation control device can determine the first gas flow rate control value and the second gas flow rate control value according to the target output flow rate and oxygen concentration setting value. That is to say, the first gas flow rate control value and the second gas flow rate control value can be set according to the target output flow rate which is set by the user and the oxygen concentration setting value which is desired by the user. Take FIG. 1 and FIG. 2 as examples, that is, the gas flow rate control value of the first gas flow rate controller 212 and the gas flow rate control value of the second gas flow rate controller 22 can be set, according to the target output flow rate which is set by the user and the oxygen concentration setting value which is desired by the user. Wherein, the target output flow rate is indirectly obtained according to a pressure set by the user.

In step 602, whether the first gas flow rate control value falls into a first dead zone range and whether the second gas flow rate control value falls into a second dead zone range are determined.

In this embodiment, after obtaining the first gas flow rate control value and the second gas flow rate control value, the ventilation control device can respectively determine whether the first gas flow rate control value falls into the first dead zone range and whether the second gas flow rate control value falls into the second dead zone range. Wherein, the first dead zone range corresponds to the dead zone range of the first gas flow rate controller, the second dead zone range corresponds to the dead zone range of the second gas flow rate control, that is, the first dead zone range is the dead zone range of the first gas flow rate controller, and the second dead zone range is the dead zone range of the second gas flow rate controller.

It should be noted that when determining whether the first gas flow rate control value falls into the first dead zone range and whether the second gas flow rate control value falls into the second dead zone range, the determination results are divided into three situations.

1. The first gas flow rate control value falls into the first dead zone range.

2. The second gas flow rate control value falls into the second dead zone range.

3. The first gas flow rate control value falls outside the first dead zone, and the second gas flow rate control value falls outside the second dead zone.

When the first gas flow rate control value falls into the first dead zone range, step 603 is executed, when the second gas flow rate control falls into the second dead zone range, step 604 is executed, when the first gas flow rate control value falls outside the first dead zone range and the second gas flow rate control value falls outside the second dead zone range, step 605 is executed.

It should be noted that before determining whether the first gas flow rate control value falls into the first dead zone range and whether the second gas flow rate control value falls into the second dead zone range, the first dead zone range corresponding to the first gas flow rate controller 212 and the second dead zone range corresponding to the second gas flow rate controller 222 need to be determined respectively. The following details are given.

In one embodiment, the ventilation control device can obtain a flow rate-electric current curve of the first gas flow rate controller and a flow rate-electric current curve of the second gas flow rate controller, and then determine the first dead zone range according to the flow rate-electric current curve of the first gas flow rate controller and determine the second dead zone range according to the flow rate-electric current curve of the second gas flow rate controller.

In this embodiment, the flow rate-electric current curve of the first gas flow rate controller can be obtained by flow calibration of the first gas flow rate controller, or by looking up the performance manual corresponding to the first gas flow rate controller provided by the manufacturer. The first dead zone range can also be set as a flow rate at the inflection point between the dead zone and linear part of the first gas flow rate controller, or of course can be set according to the actual situation. The second dead zone range corresponding to the second gas flow rate controller is also obtained in the same way.

In step 603, if the first gas flow rate control value falls into the first dead zone, the first gas flow rate controller maintains turn-on.

In this embodiment, when the first gas flow rate control value falls into the first dead zone range, the first gas control flow rate controller maintains turn-on. Here, it may include that the first gas control flow rate controller maintains turn-on in both the inspiratory phase and/or the expiratory phase.

It can be understood that when the first gas flow rate control value falls into the first dead zone range, there are also two situations. The first situation is that the first gas flow rate control value falls into the first dead zone range, and the second gas flow rate control value falls outside the second dead zone range. The second situation is that the first gas flow rate control value falls into the first dead zone range, and the second gas flow rate control value falls into the second dead zone range. Two situations are described as follows.

1. The first gas flow rate control value falls into the first dead zone range, while the second gas flow rate control value falls outside the second dead zone range.

If the first gas flow rate control value falls into the first dead zone range and the second gas flow rate control value falls outside the second dead zone range, the second gas flow rate controller is controlled to generate high-frequency oscillation.

2. The first gas flow rate control value falls into the first dead zone range, and the second gas flow rate control value also falls into the second dead zone range.

If the first gas flow rate control value falls into the first dead zone range and the second gas flow rate control value falls into the second dead zone range, the high-frequency pressure reduction module is controlled to generate high-frequency oscillation. At this time, the second gas control flow rate controller can also maintain to be turned on in the inspiratory phase and/or the expiratory phase. With reference to FIG. 1 and FIG. 2, the ventilation control device 3 can control the high-frequency pressure reduction module 4 to adjust, according to a preset high-frequency oscillation frequency, a gas in the inspiratory branch or the expiratory branch to generate high-frequency oscillation. That is, by adjusting the high-frequency valve 41 and the turbine 42, the gas in the inspiratory branch or the expiratory branch can generate high-frequency oscillation.

It should be noted that the high-frequency pressure reduction module includes a high-frequency valve and turbine. It can be seen from FIG. 1 and FIG. 2 that the high-frequency pressure reduction module 4 includes high-frequency valve 41 and turbine 42. Specifically, the high-frequency valve 41 can be any one of a proportional solenoid valve, block valve and servo valve.

In step 604, if the second gas flow rate control value falls into the second dead zone, the second gas flow rate controller maintains turn-on.

In this embodiment, when the second gas flow rate control value falls into the second dead zone range, the second gas flow rate controller is maintained turn-on. As discussed above, it may include maintaining the second gas control flow rate controller to be turned on in both the inspiratory phase and/or the expiratory phase.

It should be noted that when the second gas flow rate control value falls into the second dead zone, the comparison results between the first gas flow rate control value and the first dead zone range include two situations: 1, the first gas flow rate control value falls into the first dead zone range; 2. the first gas flow rate control value falls outside the first dead zone range, which are described below.

1. The second gas flow rate control value falls into the second dead zone range, and the first gas flow rate control value falls into the first dead zone range.

In step 603 above, the execution situation in which the second gas flow rate control value falls into the second dead zone and the first gas flow rate control value falls into the first dead zone, has been described, which is not repeated here.

2. The second gas flow rate control value falls into the second dead zone range, while the first gas flow rate control value falls outside the first dead zone range.

When the first gas flow rate control value falls outside the first dead zone range and the second gas flow rate control value falls into the second dead zone range, the first gas flow rate controller is controlled to generate high-frequency oscillation. That is to say, during comparing the first gas flow rate control value with the first dead zone range, and comparing the second gas flow rate control value with the second dead zone range, when one of the gas flow rate control values falls into its corresponding dead zone range, and the other one gas flow rate control value fall outside its corresponding dead zone range, the gas flow rate controller which corresponds to the gas flow rate control value that falls outside the corresponding dead zone range, is controlled to generate high-frequency oscillation. That is, when the target flow rate of the first gas flow rate controller 212 or the second gas flow rate controller 222 is adjusted to its corresponding dead zone range, the control flow rate is maintained without closing the valve in the inspiratory phase and/or the expiratory phase.

In step 605, if the first gas flow rate control value falls outside the first dead zone range and the second gas flow rate control value falls outside the second dead zone range, other operations are performed.

In this embodiment, if the first gas flow rate control value falls outside the first dead zone range and the second gas flow rate control value falls outside the second dead zone range, the first gas flow rate controller and the second gas flow rate controller are controlled to generate high-frequency pulse flow rate. That is to say, when the two gas flow rate control values fall outside their corresponding dead zones, the first gas flow rate controller and the second gas flow rate controller are respectively controlled to turn on and off in high-frequency pulse mode (that is, the first gas flow rate controller and the second gas flow rate controller are controlled to turn on and off continuously) to form high-frequency pulse flow rate and enhance the high-frequency oscillation of inspiratory branch 2. In addition, when the first gas flow rate control value falls outside the first dead zone and the second gas flow rate control value falls outside the second dead zone, high-frequency oscillation can also be generated through the high-frequency pressure reduction module, which is not limited herein.

It should be noted that the high-frequency pressure reduction module is adjusted according to the control demand of negative pressure. The high-frequency oscillation is completed by the pulse gas flows which are formed in the expiratory phase and the inspiratory phase. The high-frequency pressure reduction module can be configured to assist a pressure reduction in the whole inspiratory phase and the expiratory phase, especially during the generation of the negative pressure. No matter in the expiratory phase or the inspiratory phase, the high-frequency oscillation can be generated by controlling the high-frequency pressure reduction module.

In one embodiment, the inspiratory branch is also provided with an oxygen concentration detector to detect the oxygen concentration of output gas of the inspiratory branch.

If the oxygen concentration of the output gas detected by the oxygen concentration detector fails to reach the target oxygen concentration, the first gas flow rate controller and the second gas flow rate controller are adjusted according to the oxygen concentration of the output gas and the target oxygen concentration.

Combining FIG. 1 and FIG. 2 for explanation, the oxygen concentration detector can be arranged between the humidifier and the third pressure sensor 7 at the mixing branch 23 of the inspiratory branch 2, such that it can detect whether the oxygen concentration of the output gas of the whole inspiratory branch 2 reaches the target oxygen concentration. When the oxygen concentration of the output gas of the inspiratory branch 2 fails to reach the target oxygen concentration, the flow rates of the first gas flow rate controller and the second gas flow rate controller are adjusted according to the oxygen concentration of the output gas and the target oxygen concentration. For example, the flow rates of the first gas flow rate controller and the second gas flow rate controller are increased or decreased according to the oxygen concentration of the output gas and the target oxygen concentration. Specifically, a mapping relationship is maintained to implement the adjustment. The mapping relationship refers to a mapping relationship between the oxygen concentration of the output gas and the target oxygen concentration, and the flow rate of the first gas flow rate controller and the flow rate of the second gas flow rate controller.

It can be understood that when adjusting the first gas flow rate controller and the second gas flow rate controller according to the oxygen concentration and target oxygen concentration of the output gas, the first gas flow rate controller and the second gas flow rate controller can be adjusted according to the oxygen concentration and target oxygen concentration of the output gas, based on a preset adjustment rule.

That is to say, an adjustment step length, adjustment frequency or adjustment period can be preset in advance to adjust the first gas flow rate controller and the second gas flow rate controller according to the oxygen concentration of the output gas and the target oxygen concentration. For example, the first gas flow rate controller and the second gas flow rate controller are adjusted in each expiratory phase and inspiratory phase. Further, the first gas flow rate controller and the second gas flow rate controller can also be adjusted each two expiratory phases and each two inspiratory phases. Of course, other adjustment rules can also be adopted for adjusting, such as adjusting the first gas flow rate controller and the second gas flow rate controller by taking two seconds as a period, or adjusting the first gas flow rate controller and the second gas flow rate controller once for each two seconds. Of course, they can also be adjusted according to the actual situation, as long as the adjustments of the first gas flow rate controller and the second gas flow control can be realized. In such a way, a closed-loop adjustment can be formed. Whether the adjusted flow rates of the first gas flow rate controller and the second gas flow rate controller are in their respective dead zones, are determined and then subsequent operations are performed.

It should be noted that during the closed-loop adjustment, if the oxygen concentration of the output gas of inspiratory branch 2 is stabilized at the target oxygen concentration, the adjustment stops. Otherwise, the adjustment continues until the oxygen concentration of the output gas of inspiratory branch 2 is stabilized at the target oxygen concentration.

To sum up, it can be seen that in the embodiment provided by this disclosure, the first gas flow rate control value and the second gas flow rate control value are determined according to the target output flow rate and oxygen concentration settings, and whether the first gas flow rate control value falls into the first dead zone range and whether the second gas flow rate control value falls into the second dead zone range are determined respectively. If the first gas flow rate control value falls into the first dead zone range, the first gas flow rate controller can maintain turn-on in the inspiratory phase and/or expiratory phase; if the second gas flow rate control value falls into the second dead zone range, the second gas flow rate controller can maintain turn-on in the inspiratory phase and/or expiratory phase, so that when the flow rate of the proportional valve is within its corresponding dead zone range, a stable and small flow rate can be generated in the high-frequency oscillation process to ensure stable and accurate oxygen concentration control within the oxygen concentration setting range, through adjusting the flow rate of the proportional valve.

Please refer to FIG. 7, which is a virtual structure diagram of a high-frequency ventilation system provided by an embodiment of this disclosure. The high-frequency ventilation system 700 includes a gas source interface 701, an inspiratory branch (not shown in FIG. 7), a high-frequency pressure reduction module 702, and a ventilation control device 703. The inspiratory branch includes a first gas branch, a second gas branch, and a mixing branch, a first gas flow rate controller which generates a high-frequency pulse flow rate and is arranged at the first gas branch, and a second gas flow rate controller which generates a high-frequency pulse flow rate and is arranged at the second gas branch; wherein the ventilation control device 703 is configured to:

determine a first gas flow rate control value and a second gas flow rate control value according to a target output flow rate and an oxygen concentration setting value;

determine whether the first gas flow rate control value falls into a first dead zone range and deteimine whether the second gas flow rate control value falls into a second dead zone range;

maintain the first gas flow rate controller turn-on, if the first gas flow rate control value falls into the first dead zone range; and

maintain the second gas flow rate controller turn-on, if the second gas flow rate control value falls into the second dead zone range;

wherein the first dead zone range corresponds to a dead zone range of the first gas flow rate controller, and the second dead zone range corresponds to a dead zone range of the second gas flow rate controller.

Optionally, the ventilation control device 703 is further configured to:

control the second gas flow rate controller to generate high-frequency oscillation, if the first gas flow rate control value falls into the first dead zone range and the second gas flow rate control value falls outside the second dead zone range;

control the first gas flow rate controller to generate high-frequency oscillation, if the first gas flow rate control value falls outside the first dead zone range and the second gas flow rate control value falls into the second dead zone range.

Optionally, the ventilation control device 703 is further configured to:

control the high-frequency pressure reduction module to generate high-frequency oscillation, if the first gas flow rate control value falls into the first dead zone range and the second gas flow rate control value falls into the second dead zone range.

Optionally, the high-frequency pressure reduction module 702 includes a high-frequency valve and a turbine.

Optionally, in order to control the high-frequency pressure reduction module to generate high-frequency oscillation, the ventilation control device 703 is further configured to:

control the high-frequency valve and the turbine to generate the high-frequency oscillation according to a preset high-frequency oscillation frequency.

Optionally, the ventilation control device 703 is further configured to:

control the first gas flow rate controller and the second gas flow rate controller to generate a high-frequency pulse flow rate, if the first gas flow rate control value falls outside the first dead zone range and the second gas flow rate control value falls outside the second dead zone range.

Optionally, the inspiratory branch is further provided with an oxygen concentration detector, which is configured to detect an oxygen concentration of an output gas of the inspiratory branch; wherein the ventilation control device 703 is further configured to:

adjust the first gas flow rate controller and the second gas flow rate controller according to the oxygen concentration of the output gas and a target oxygen concentration, if the oxygen concentration of the output gas which is detected by the oxygen concentration detector fails to reach the target oxygen concentration.

Optionally, in order to adjust the first gas flow rate controller and the second gas flow rate controller according to the oxygen concentration of the output gas and a target oxygen concentration, the ventilation control device 703 is further configured to:

adjust the first gas flow rate controller and the second gas flow rate controller according to the oxygen concentration of the output gas and the target oxygen concentration, based on a preset adjustment rule.

Optionally, the ventilation control device 703 is further configured to:

determine the first dead zone range according to a flow rate-electric current curve of the first gas flow rate controller; and

determine the second dead zone range according to a flow rate-electric current curve of the second gas flow rate controller.

Optionally, the ventilation control device 703 is further configured to:

obtain the flow rate-electric current curve of the first gas flow rate controller and the flow rate-electric current curve of the second gas flow rate controller.

Of course, the arrangements and controls of the gas source interface 701, the high-frequency pressure reduction module 702, the inspiratory branch, the expiratory branch, the first gas flow rate controller, the second gas flow rate controller, etc., in the high-frequency ventilation system can refer to FIGS. 1, 2 and the above descriptions, which is not repeated here.

To sum up, it can be seen that in the embodiment provided by this disclosure, the first gas flow rate control value and the second gas flow rate control value are determined according to the target output flow rate and oxygen concentration setting, whether the first gas flow rate control value falls into the first dead zone range and whether the second gas flow rate control value falls into the second dead zone range are determined respectively, if the first gas flow rate control value falls into the first dead zone, the first gas flow rate controller maintains turn-on in the expiratory phase, while if the second gas flow rate control value falls into the second dead zone, the second gas flow rate controller maintains turn-on in the expiratory phase, so that when the flow rate of the proportional valve is within its corresponding dead zone, the flow rate of the proportional valve can be adjusted to produce a stable small flow rate in the high-frequency oscillation process, which ensures stable and accurate oxygen concentration control within the oxygen concentration setting range.

In several embodiments provided in this disclosure, it should be understood that the disclosed system, device and method can be implemented in other ways. For example, the device embodiments described above are only schematic, for example, the division of the units is only logical function divisions, and there can be another division method when actually implemented, for example, multiple units or components can be combined or integrated into another system, or some features can be ignored or not implemented. On the other hand, the mutual coupling or direct coupling or communication connection shown or discussed can be indirect coupling or communication connection through some interfaces, devices or units, and can be electrical, mechanical or other forms.

The units described as separate units may or may not be physically separated, and the components which are displayed as units may or may not be physical units, that is, they may be located in one place or distributed to multiple network units. Some or all of the units can be selected according to actual requirements to achieve the purpose of the embodiment.

In addition, each functional unit in each embodiment of this disclosure can be integrated in a processing unit, or each unit can exist physically independently, or two or more units can be integrated in one unit. The integrated units mentioned above can be realized in the form of hardware or software functional units.

If the integrated unit is realized in the form of a software functional unit and sold or used as an independent product, it can be stored in a computer readable storage medium. Based on this understanding, the technical solution of this disclosure, in essence, or the part that contributes to the prior art, or the whole or part of the technical solution, can be embodied in the form of a software product, which is stored in a storage medium, including a number of instructions to enable a computer device (which may be a personal computer, a server, or a network device, etc.) to perform all or part of the steps of the method described in various embodiments of this disclosure. The aforementioned storage media include U disk, mobile hard disk, read only memory (ROM), random access memory (RAM), magnetic disc or optical disc and other media that can store program codes.

The above description is only the specific implementation mode of this disclosure, but the protection scope of this disclosure is not limited to this. Any person skilled in the technical field can easily recognize that changes or replacements can be made to the details of the above embodiments without departing from the basic principles of this disclosure. Therefore, the scope of this disclosure shall be determined according to the following attached claims.

What is claimed is:

1. A ventilation adjustment method applied to a high-frequency ventilation system, wherein the high-frequency ventilation system comprises a gas source interface, an inspiratory branch, a ventilation control device, and a high-frequency pressure reduction module, wherein the inspiratory branch comprises a first gas branch, a second gas branch, a mixing branch, a first gas flow rate controller that generates a high-frequency pulse flow rate and is arranged at the first gas branch, and a second gas flow rate controller that generates a high-frequency pulse flow rate and is arranged at the second gas branch,
wherein the ventilation adjustment method comprises:
determining a first gas flow rate control value and a second gas flow rate control value according to a target output flow rate and an oxygen concentration setting value;
determining whether the first gas flow rate control value falls into a first designated range and determining whether the second gas flow rate control value falls into a second designated range;
maintaining the first gas flow rate controller turned on, if the first gas flow rate control value falls into the first designated range; and
maintaining the second gas flow rate controller turned on, if the second gas flow rate control value falls into the second designated range.

2. The ventilation adjustment method according to claim 1, wherein the first designated range corresponds to a first dead zone range of the first gas flow rate controller and the second designated range corresponds to a second dead zone range of the second gas flow rate controller.

3. The ventilation adjustment method according to claim 1, wherein the first designated range corresponds to a flow rate range in which a flow rate, a flow amount or an oxygen concentration of the first gas branch falls outside a designated range, the second designated range corresponds to a flow rate range in which a flow rate, a flow amount or an oxygen concentration of the second gas branch falls outside a designated range.

4. The ventilation adjustment method according to claim 2, wherein the ventilation adjustment method further comprises:
controlling the second gas flow rate controller to generate high-frequency oscillation, if the first gas flow rate control value falls into the first dead zone range and the second gas flow rate control value falls outside the second dead zone range; and
controlling the first gas flow rate controller to generate high-frequency oscillation, if the first gas flow rate control value falls outside the first dead zone range and the second gas flow rate control value falls into the second dead zone range.

5. The ventilation adjustment method according to claim 2, wherein the ventilation adjustment method further comprises:
controlling the high-frequency pressure reduction module to generate high-frequency oscillation, if the first gas flow rate control value falls into the first dead zone range and the second gas flow rate control value falls into the second dead zone range.

6. The ventilation adjustment method according to claim 5, wherein the high-frequency pressure reduction module comprises a high-frequency valve and a turbine;
wherein controlling the high-frequency pressure reduction module to generate high-frequency oscillation, comprises:
controlling the high-frequency valve and the turbine to generate the high-frequency oscillation according to a preset high-frequency oscillation frequency.

7. The ventilation adjustment method according to claim 2, wherein the ventilation adjustment method further comprises:
controlling the first gas flow rate controller and the second gas flow rate controller to generate respective high-frequency pulse flow rates, if the first gas flow rate control value falls outside the first dead zone range and the second gas flow rate control value falls outside the second dead zone range.

8. The ventilation adjustment method according to claim 2, wherein the inspiratory branch is further provided with an oxygen concentration detector, which is configured to detect an oxygen concentration of an output gas of the inspiratory branch;
wherein the ventilation adjustment method further comprises:
adjusting the first gas flow rate controller and the second gas flow rate controller according to the oxygen concentration of the output gas and a target oxygen concentration, if the oxygen concentration of the output gas which is detected by the oxygen concentration detector fails to reach the target oxygen concentration.

9. The ventilation adjustment method according to claim 8, wherein adjusting the first gas flow rate controller and the second gas flow rate controller according to the oxygen concentration of the output gas and a target oxygen concentration, comprises:
adjusting the first gas flow rate controller and the second gas flow rate controller according to the oxygen concentration of the output gas and the target oxygen concentration, based on a preset adjustment rule.

10. The ventilation adjustment method according to claim 2, wherein the ventilation adjustment method further comprises:
determining the first dead zone range according to a flow rate-electric current curve of the first gas flow rate controller; and
determining the second dead zone range according to a flow rate-electric current curve of the second gas flow rate controller.

11. The ventilation adjustment method according to claim 10, wherein the ventilation adjustment method further comprises:
obtaining the flow rate-electric current curve of the first gas flow rate controller and the flow rate-electric current curve of the second gas flow rate controller.

12. A high-frequency ventilation system comprising:
a gas source interface;
an inspiratory branch;
a ventilation control device; and
a high-frequency pressure reduction module,
wherein the inspiratory branch comprises: a first gas branch, a second gas branch, a mixing branch, a first gas flow rate controller which generates a high-frequency pulse flow rate and is arranged at the first gas branch, and a second gas flow rate controller which generates a high-frequency pulse flow rate and is arranged at the second gas branch,
wherein the ventilation control device is configured to:
determine a first gas flow rate control value and a second gas flow rate control value according to a target output flow rate and an oxygen concentration setting value;
determine whether the first gas flow rate control value falls into a first designated range and deteimine whether the second gas flow rate control value falls into a second designated range;
maintain the first gas flow rate controller turned on, if the first gas flow rate control value falls into the first designated range; and
maintain the second gas flow rate controller turned on, if the second gas flow rate control value falls into the second designated range.

13. The high-frequency ventilation system according to claim 12, wherein the first designated range corresponds to a first dead zone range of the first gas flow rate controller, and the second designated range corresponds to a second dead zone range of the second gas flow rate controller.

14. The high-frequency ventilation system according to claim 12, wherein the first designated range corresponds to a flow rate range in which a flow rate, a flow amount or an oxygen concentration of the first gas branch falls outside a designated range, the second designated range corresponds to a flow rate range in which a flow rate, a flow amount or an oxygen concentration of the second gas branch falls outside a designated range.

15. The high-frequency ventilation system according to claim 13, wherein the ventilation control device is further configured to:
control the second gas flow rate controller to generate high-frequency oscillation, if the first gas flow rate control value falls into the first dead zone range and the second gas flow rate control value falls outside the second dead zone range; and
control the first gas flow rate controller to generate high-frequency oscillation, if the first gas flow rate control value falls outside the first dead zone range and the second gas flow rate control value falls into the second dead zone range.

16. The high-frequency ventilation system according to claim 13, wherein the ventilation control device is further configured to:
control the high-frequency pressure reduction module to generate high-frequency oscillation, if the first gas flow rate control value falls into the first dead zone range and the second gas flow rate control value falls into the second dead zone range.

17. The high-frequency ventilation system according to claim 16, wherein the high-frequency pressure reduction module comprises a high-frequency valve and a turbine;
the ventilation control device is further configured to:
control the high-frequency valve and the turbine to generate the high-frequency oscillation according to a preset high-frequency oscillation frequency.

18. The high-frequency ventilation system according to claim 13, wherein the ventilation control device is further configured to:
control the first gas flow rate controller and the second gas flow rate controller to generate respective high-frequency pulse flow rates, if the first gas flow rate control value falls outside the first dead zone range and the second gas flow rate control value falls outside the second dead zone range.

19. The high-frequency ventilation system according to claim 13, wherein the inspiratory branch is further provided with an oxygen concentration detector, which is configured to detect an oxygen concentration of an output gas of the inspiratory branch;
wherein the ventilation control device is further configured to:
adjust the first gas flow rate controller and the second gas flow rate controller based on a preset adjustment rule, according to the oxygen concentration of the output gas and a target oxygen concentration, if the oxygen concentration of the output gas which is detected by the oxygen concentration detector fails to reach the target oxygen concentration.

20. The high-frequency ventilation system according to claim 13, wherein the ventilation control device is further configured to:
obtain a flow rate-electric current curve of the first gas flow rate controller and a flow rate-electric current curve of the second gas flow rate controller;
determine the first dead zone range according to the flow rate-electric current curve of the first gas flow rate controller; and
determine the second dead zone range according to the flow rate-electric current curve of the second gas flow rate controller.
